(19) United States
(12) Patent Application Publication
(10) Pub. No.: US 2022/0219010 A1
(43) Pub. Date: Jul. 14, 2022

(54) OPTICAL MASK DEVICE AND CONTROL METHOD THEREFOR

(71) Applicant: SAMSUNG ELECTRONICS CO., LTD., Suwon-si (KR)

(72) Inventor: Taeduk KIM, Suwon-si (KR)

(73) Assignee: SAMSUNG ELECTRONICS CO., LTD., Suwon-si (KR)

(21) Appl. No.: 17/708,824

(22) Filed: Mar. 30, 2022

Related U.S. Application Data
(63) Continuation of application No. PCT/KR2020/013715, filed on Oct. 8, 2020.

(30) Foreign Application Priority Data
Oct. 31, 2019 (KR) 10-2019-0138148

(51) Int. Cl. A61N 5/06; G16H 20/40; G16H 40/67; G01K 3/00

(52) U.S. Cl. CPC A61N 5/0616; G16H 20/40; G16H 40/67; A61N 2005/0663; A61N 2005/0629; A61N 2005/0647; A61N 2005/0652; G01K 3/005

(57) ABSTRACT

An optical mask device is disclosed. The optical mask device comprises: a main body having a face shape and to which a plurality of light-emitting diode (LED) light sources are attached; a proximity sensor; and a processor for identifying whether the main body is worn on the face of a user on the basis of a signal acquired by the proximity sensor when a skin care mode is determined, and providing a light guide corresponding to the skin care mode by emission, by less than a threshold intensity, of at least some LEDs corresponding to the skin care mode, among a plurality of LEDs, when the main body is identified not to be worn on the face of the user.

### CROSS REFERENCE TO THE RELATED APPLICATION

This application is a continuation application, under 35 U.S.C. § 111(a), of international application No. PCT/KR2020/013715, filed Oct. 8, 2020 which claims priority to Korean patent application No. 10-2019-0138148, filed Oct. 31, 2019, the entire disclosures of all of which are herein incorporated by reference as a part of this application.

### BACKGROUND

### Field

This disclosure relates to an optical mask device and a control method therefor and, more particularly, to an optical mask device providing a plurality of skin care modes and a control method therefor.

### Description of the Related Art

With the development of electronic technology, various types of electronic devices are being developed and distributed. In particular, in recent years, a skin care device that may be easily used at home, for example, a light-emitting diode (LED) mask has been developed and available in the market.

However, in the case of the currently marketed LED mask, there is a problem in that the LED mask may not provide an optimized skin care to a user due to a fixed type of LED and a predetermined quantity of LED light, or the like. Also, there is a problem that the user may not be provided with a guide on what kind of skin care the user may get.

### SUMMARY

It is an objective of the disclosure to provide an optical mask device for visually providing a guide for a skin care mode and a control method thereof.

According to an embodiment, an optical mask device includes a main body having a face shape and wearable on a face of a user, a plurality of light-emitting diode (LED) light sources provided in the main body, a proximity sensor to send a signal in response to sensing a presence of the face of the user, and a processor configured to receive a skin care mode selected by the user, identify whether the main body is worn on the face of the user based on the signal received from the proximity sensor, and in response to the identifying that the main body is not worn on the face of the user, provide a light guide corresponding to the skin care mode by controlling at least some LEDs corresponding to the skin care mode among a plurality of LEDs to emit an intensity less than a threshold intensity.

The processor may, in response to the identifying that the main body is worn on the face of the user while providing the light guide, control the optical mask device to operate in the skin care mode emitting the at least some LEDs with an intensity greater than or equal to a threshold intensity.

The processor may emit the at least some LEDs with the intensity less than the threshold intensity by controlling a duty of a current provided to the at least some LEDs with a value less than a threshold value, and emit the at least some LEDs with the intensity greater than or equal to the threshold intensity by controlling the duty of the current provided to the at least some LEDs with a value greater than or equal to the threshold value.

The optical mask device may further include a temperature sensor, and the processor may, based on a value sensed by the temperature sensor while operating in the skin care mode being greater than or equal to a threshold temperature value, reduce the duty of the current provided to the at least some LEDs.

The processor may provide a light guide corresponding to the skin care mode by emitting the at least some LEDs corresponding to the skin care mode among the plurality of LEDs with the intensity less than the threshold intensity.

The processor may provide a light guide corresponding to the skin care mode by sequentially emitting LEDs in different colors corresponding to the skin care mode among the plurality of LEDs with the intensity less than the threshold intensity.

The processor may, based on emission, by greater than or equal to the threshold intensity, of the LED in a first color for a first time in the skin care mode and emission, by greater than or equal to the threshold intensity, of the LED in a second color for a second time that is shorter than the first time, provide the light guide so that an LED of the first color and an LED of the second color emit light by less than the threshold intensity, by a time corresponding to a ratio of the first time and the second time within a preset time.

The optical mask device may further include a memory storing usage history of a skin care mode by the user, and the processor may, based on the optical mask device being turned on, automatically determine the skin care mode based on the stored usage history of a skin care mode of the user.

The processor may automatically determine the skin care mode based on skin state information of the user received from an external device.

The skin care mode may include a normal care mode or a speedy care mode divided by a skin care time.

The skin care mode may include at least one of an elasticity care mode, a trouble care mode, a whitening care mode, or a wrinkle care mode divided by a skin care type.

According to an embodiment, a control method of an optical mask device comprising a main body having a face shape and wearable on a face of a user, a plurality of light-emitting diode light sources provided in the main body, and a proximity sensor may send a signal in response to sensing a presence of the face of the user, the method may include determining a skin care mode for the face of the user, identifying whether the main body is worn on the face of the user based on the signal received from the proximity sensor, and in response to the identifying that the main body is not worn on the face of the user, providing a light guide corresponding to the skin care mode by controlling at least some LEDs corresponding to the skin care mode among a plurality of LEDs to emit an intensity less than a threshold intensity.

The method may further include, in response to the identifying that the main body is worn on the face of the user, operating in the skin care mode by emitting the at least some LEDs with an intensity greater than or equal to a threshold intensity.

The providing the light guide may further include emitting the at least some LEDs with the intensity less than the threshold intensity by controlling a duty of a current provided to the at least some LEDs with a value less than a threshold value, and the operating in the skin care mode may further include emitting the at least some LEDs with the intensity greater than or equal to the threshold intensity by controlling the duty of the current provided to the at least some LEDs with a value greater than or equal to the threshold value.

The optical mask device may further include a temperature sensor, and the method may further include, based on a value sensed by the temperature sensor while operating in the skin care mode being greater than or equal to a threshold temperature value, reducing the duty of the current provided to the at least some LEDs.

The providing the light guide may include providing a light guide corresponding to the skin care mode by emission, by less than the threshold intensity, of at least some LEDs corresponding to the skin care mode, among the plurality of LEDs.

The providing the light guide may include providing a light guide corresponding to the skin care mode by sequential emission, by less than the threshold intensity, of LEDs in different colors corresponding to the skin care mode, among the plurality of LEDs.

The providing the light guide may include, based on emission, by greater than or equal to the threshold intensity, of the LED in a first color for a first time in the skin care mode and emission, by greater than or equal to the threshold intensity, of the LED in a second color for a second time that is shorter than the first time, providing the light guide so that an LED of the first color and an LED of the second color emit light by less than the threshold intensity, by a time corresponding to a ratio of the first time and the second time within a preset time.

The method further include, based on the optical mask device being turned on, automatically determining the skin care mode based on the usage history of a skin care mode of the user.

The method may further include automatically determining the skin care mode based on skin state information of the user received from an external device.

According to the above-described various embodiments, it is possible to minimize the power consumption of the LED before a user wearing the optical mask while providing information on the skin care mode to the user by using the LED provided in the optical mask.

### BRIEF DESCRIPTION OF DRAWINGS

FIG. 1 is a diagram illustrating a configuration of a skin care system according to an embodiment of the disclosure;

FIG. 2 is a block diagram illustrating a configuration of an optical mask device according to an embodiment of the disclosure;

FIG. 3 is a diagram illustrating an operation of a processor 130 according to an embodiment of the disclosure;

FIG. 4 is a diagram illustrating an operation of a processor 130 of FIG. 3 according to an embodiment of the disclosure in greater detail;

FIG. 5 is a diagram illustrating a method of providing a light guide according to an embodiment of the disclosure;

FIG. 6 is a diagram illustrating a method of controlling an LED according to skin temperature according to an embodiment of the disclosure;

FIGS. 7A and 7B are diagrams to assist understanding of a skin care mode according to an embodiment of the disclosure;

FIGS. 8A, 8B, and 9A, 9B, 9C and 9D are diagrams illustrating various skin care modes according to an embodiment of the disclosure; and

FIG. 10 is a block diagram illustrating a specific configuration of an optical mask device according to an embodiment of the disclosure.

### DETAILED DESCRIPTION

The disclosure will be described in greater detail with reference to the attached drawings.

The terms used in this specification will be briefly described, and the disclosure will be described in detail.

The terms used in the disclosure and the claims are general terms identified in consideration of the functions of embodiments of the disclosure. However, these terms may vary depending on intention, legal or technical interpretation, emergence of new technologies, and the like of those skilled in the related art. In addition, in some cases, a term may be selected by the applicant, in which case the term will be described in detail in the description of the corresponding disclosure. Thus, the term used in this disclosure should be defined based on the meaning of term, not a simple name of the term, and the contents throughout this disclosure.

As used herein, the terms “first,” “second,” or the like may identify corresponding components, and are used to distinguish a component from another without limiting the components.

A singular expression includes a plural expression, unless otherwise specified. It is to be understood that the terms such as “comprise” may, for example, be used to designate a presence of a characteristic, number, step, operation, element, component, or a combination thereof, and not to preclude a presence or a possibility of adding one or more of other characteristics, numbers, steps, operations, elements, components or a combination thereof.

Expressions such as “at least one of A or B” and “at least one of A and B” should be understood to represent “A,” “B” or “A and B.”

A term such as “module,” “unit,” and “part,” is used to refer to an element that performs at least one function or operation and that may be implemented as hardware or software, or a combination of hardware and software. Except when each of a plurality of “modules,” “units,” “parts,” and the like must be realized in an individual hardware, the components may be integrated in at least one module or chip and be realized in at least one processor (not shown).

Embodiments of the disclosure will be described in detail with reference to the accompanying drawings to aid in the understanding of those of ordinary skill in the art. However, the disclosure may be realized in various different forms and it should be noted that the disclosure is not limited to the various embodiments described herein. Further, in the drawings, parts not relevant to the description may be omitted, and like reference numerals may be used to indicate like elements.

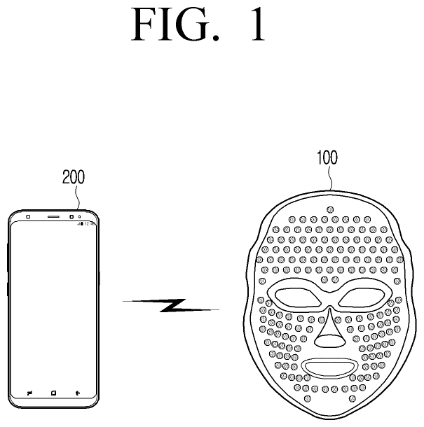

FIG. 1 is a diagram illustrating a configuration of a skin care system according to an embodiment of the disclosure.

Referring to FIG. 1, a skin care system may include an optical mask device 100 and a user terminal device 200.

The optical mask device 100 is a skin care device that is in a shape of a mask wearable on a face region of a user. Specifically, the optical mask device 100 may help treat damaged skin using a principle that the light source, for example, an LED light source, is disposed in a direction in which the product and the skin are in contact with each other, and light promotes biochemical reaction in the skin.

According to an example, a pair of LED of different types or colors may be adjacently disposed in the optical mask device 100. That is, at least two LEDs may be disposed adjacent to each other at the same position. Here, different types or colors of LED pairs may include at least one of an IR LED, a red LED, a blue LED, a green LED, or a yellow LED.

However, different types or colors of LEDs may be arranged at a ratio of 1:1, but may be arranged at a ratio of N:1 (N≥1). For example, the number of IR LEDs and the red LEDs and the number of LEDs of the remaining colors may be N:1(N≥1).

For example, when a plurality of paired LEDs disposed in the same position are composed of IR LEDs and red LEDs, only IR LEDs may be emitted depending on the skin care mode, or only red LEDs may be emitted.

The user terminal device 200 may be implemented as a cellular phone, such as a smartphone, as shown in FIG. 1, but may be implemented as various types of devices that are capable of communicating with an optical mask device 100, such as a tablet personal computer (PC), a mobile phone, a desktop personal computer (PC), a laptop personal computer (PC), a personal digital assistant (PDA), a portable multimedia player (PC), an MP3 player, a mobile medical device, a camera, a camcorder, an electronic frame, or a wearable device (e.g., a head-mounted-device (HMD)) smart watch, an electronic garment, an electronic bracelet, an electronic necklace, etc.).

According to an embodiment, the user terminal device 200 may provide a skin analysis and care service for a user face included in an image captured by a camera (not shown). The user terminal device 200 may be implemented to store various programs for providing skin analysis and care services. In particular, according to an embodiment, the skin analysis and care service may be provided in the form of an application which is software that a user directly uses on an operating system (OS), and the application may be provided in an icon interface form on a screen of the user terminal device 200. However, the skin analysis and care service may be provided in various forms depending on the type of implementation of the user terminal device 200, without being limited thereto.

The user terminal device 200 may transmit a skin analysis result for the user's face to the optical mask device 100, or transmit information about the skin care mode determined based on the skin analysis result to the optical mask device 100.

For example, the user terminal device 200 may measure and analyze various skin conditions such as pores, acne, pigmentation, skin tone, dark circles, wrinkles, and the like in a face region of a user in a captured image. The user terminal device 200 may perform a detailed capturing operation of focusing and enlarging a corresponding area in order to capture one area of a user's face in detail according to some cases. In this example, the item to be analyzed may be an item set by default when the user terminal device 200 is manufactured or an application is installed, but in some cases, the item may be an item selected by a user from among items that may be analyzed by the user terminal device 100.

According to another embodiment, the user terminal device 200 may be implemented to communicate with a server (not shown) through network to provide skin analysis and care services.

When the user terminal device 200 transmits a user image for skin analysis to a server (not shown), the server (not shown) may perform skin analysis on the user face region included in the user image, and may provide the result to the user terminal device 200. In this case, a server (not shown) may be implemented to perform a function related to skin analysis. The server (not shown) may be implemented as a cloud server, but is not limited thereto. Cloud computing refers to a cloud-based computing technology, for example, a software service based on a web used and retrieved to a computer, a mobile phone, or the like, from a utility data server on the Internet. The server (not shown) may be implemented as an external server or an embedded server provided in the user terminal device 200 according to a physical implementation form.

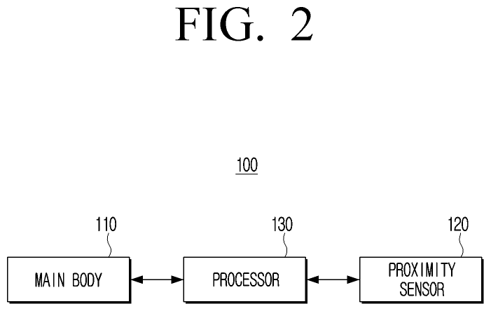

FIG. 2 is a block diagram illustrating a configuration of an optical mask device according to an embodiment of the disclosure.

Referring to FIG. 2, the optical mask device 100 includes a main body 110, a proximity sensor 120, and a processor 130.

The main body 110 is formed in the shape of a face of a human, and a plurality of light sources (111) may be attached to the surface of the body 110 in the direction of being in contact with the skin. The plurality of light sources (111) may be a LED light source, but is not limited thereto, and may be implemented as various types of light sources suitable for skin care.

A proximity sensor 120 is a sensor capable of sensing the presence of an ambient object without physical contact. The proximity sensor 120 according to an embodiment of the disclosure may be provided in one region of the main body 110 of the optical mask device 100 and may sense whether the optical mask device 100 is worn on the face of the user. For example, the proximity sensor 120 may be implemented as at least one of a high frequency oscillation type proximity sensor, an optical fiber proximity sensor, an electromagnetic proximity sensor, a capacitive proximity sensor, an optical proximity sensor, or an ultrasonic proximity sensor.

The processor 130 is electrically connected to the proximity sensor 120 to control the overall operation of the optical mask device 100. The processor 130 may be configured with one or a plurality of processors. For example, the processor 130 may perform an operation of the optical mask device 100 according to various embodiments of the disclosure by executing at least one instruction stored in a memory (not shown).

The processor 130 according to an embodiment may be implemented with, for example, and without limitation, a digital signal processor (DSP) for processing of a digital signal, a microprocessor, a graphics processing unit (GPU), an artificial intelligence (AI) processor, a neural processing unit (NPU), a time controller (TCON), or the like. The processor 130 may include, for example, and without limitation, one or more among a central processor (CPU), a micro controller unit (MCU), a micro processor (MPU), a controller, an application processor (AP), a communication processor (CP), an advanced reduced instruction set computing (RISC) machine (ARM) processor, a dedicated processor, or may be defined as a corresponding term. The processor 130 may be implemented in a system on chip (SoC) type or a large scale integration (LSI) type which a processing algorithm is built therein, application specific integrated circuit (ASIC), or in a field programmable gate array (FPGA) type.

When the optical mask device 100 is implemented to execute an artificial intelligence (AI) model according to an example, the processor 130 for executing the AI model may be a general purpose processor such as a central processing unit (CPU), an application processor (AP), a digital signal processor (DSP), a graphics-only processor such as graphics processing unit (GPU), a vision processing unit (VPU), or an AI-dedicated processor such as a neural processing unit (NPU). The processor 130 may control to process the input data in accordance with a predefined operating rule or an artificial intelligence model stored in a memory (not shown). Alternatively, if the processor 130 is an AI dedicated processor, it may be designed in a hardware structure specialized for processing a particular AI model. For example, hardware specialized for processing a particular AI model may be designed as hardware chips, such as an application-specific integrated circuit (ASIC), a field-programmable gate array (FPGA), and the like.

According to an embodiment of the disclosure, when the power of the optical mask device 100 is turned on, the processor 130 may identify whether the main body 110 is worn on the face of the user based on the signal obtained by the proximity sensor 120, and may adjust the intensity of the plurality of LEDs differently depending on whether the main body 110 is worn. For example, if the body 110 is identified as not being worn on the face of the user, the plurality of LEDs may be emitted below the threshold intensity.

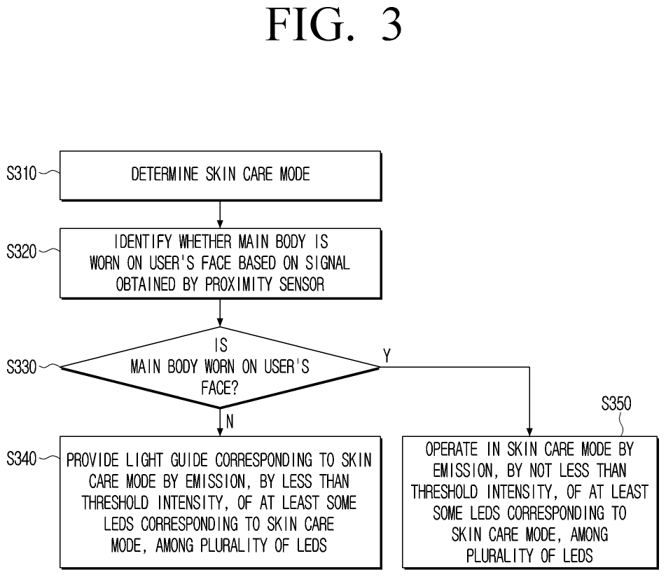

FIG. 3 is a diagram illustrating an operation of a processor 130 according to an embodiment of the disclosure.

According to FIG. 3, when the skin care mode is determined in operation S310, the processor 130 may identify whether the main body 110 is worn on the user's face based on the signal obtained by the proximity sensor 120 in operation S320.

Based on the main body being identified not to be worn on the face of the user in operation S330, the processor 130 may provide a light guide corresponding to the skin care mode by emission, by less than a threshold intensity, of at least some LEDs corresponding to the skin care mode, among a plurality of LEDs in operation S340. That is, it is possible to provide information about the skin care mode that the user will get while preventing glare to the user's eyes.

If the main body 110 is identified as being worn on the face of the user in operation S330, the processor 130 may emit at least a part of LED light corresponding to the skin care mode among the plurality of LEDs by a threshold intensity or more and may operate in the skin care mode in operation S350.

In this example, the processor 130 may control the duty of the current provided to at least some LEDs corresponding to the skin care mode to less than the threshold value to emit the LED below the threshold intensity. Further, the processor 130 may control the duty of the current provided to at least some LEDs corresponding to the skin care mode above a threshold value to emit at least some LEDs above a threshold intensity.

The processor 130 may adjust supply time of the driving current (or driving voltage) supplied to the plurality of LEDs. The processor 130 may control light emitting intensity of a plurality of LEDs by pulse width modulation (PWM) with a variable duty ratio. Here, the PWM signal may control the lighting and lights-out ratio of the light sources, and the duty ratio (%) is determined according to the dimming value input from the processor 130. In some cases, the intensity of the driving current (or driving voltage) supplied to the plurality of LEDs is possible.

In this case, the processor 130 may be implemented to include a driver integrated circuit (IC) for driving a plurality of LEDs. For example, the processor 130 may be implemented as a digital signal processor (DSP) and implemented with a digital driver IC as one chip. However, it is needless to say that the driver IC may be implemented by hardware separate from the processor 130. For example, when the light sources included in a backlight unit (not shown) are implemented as LED devices, the driver IC may be implemented with at least one LED driver that controls a current applied to the LED. According to one embodiment, the LED driver may be disposed at the rear end of a power supply (for example, a switching mode power supply (SMPS)) to receive a voltage from the power supply. However, according to another embodiment, a voltage may be applied from a separate power supply device. Alternatively, it is also possible that the SMPS and LED drivers are implemented in one integrated module.

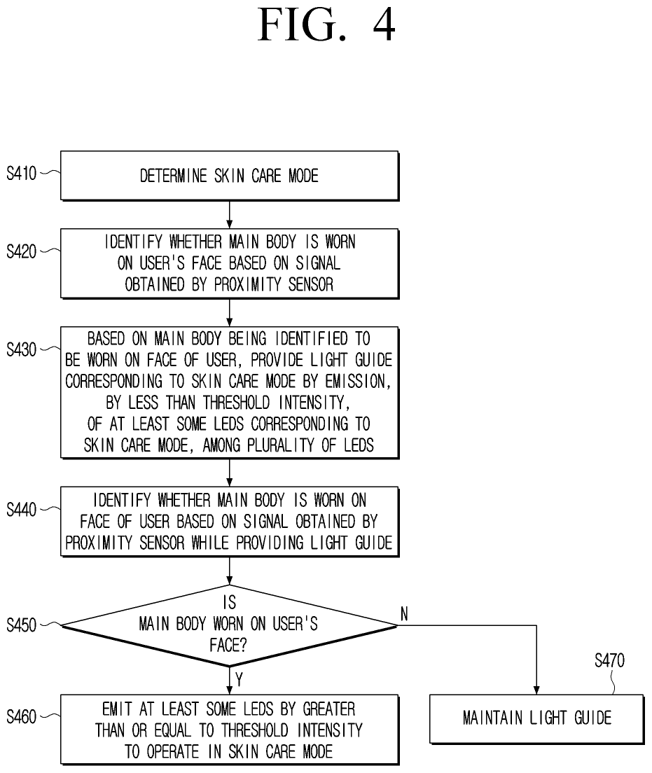

FIG. 4 is a diagram illustrating an operation of the processor 130 of FIG. 3 according to an embodiment of the disclosure in greater detail.

Referring to FIG. 4, in operation S420, the processor 130 may identify whether the main body 110 is worn on the face of a user on a basis of a signal obtained by the proximity sensor 120 based on a skin care mode being determined in operation S410.

Based on the main body 110 being identified not to be worn on the face of the user, the processor 130 may provide a light guide corresponding to the skin care mode by emission, by less than a threshold intensity, of at least some LEDs corresponding to the skin care mode, among a plurality of LEDs in operation S430.

In operation S440, the processor 130 may identify that the main body 110 is worn on the face of the user based on a sensing signal obtained by the proximity sensor 120 while providing the light guide in operation S430.

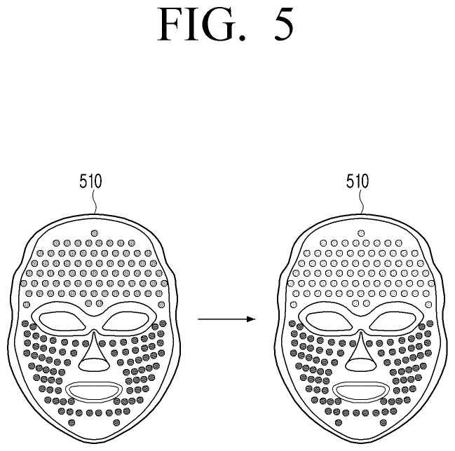

Based on identifying that the main body 110 is worn on the face of the user in operation S450, the processor 130 may emit at least some LEDs corresponding to the light guide, that is, at least some LEDs corresponding to the skin care mode by greater than or equal to a threshold intensity to operate in the skin care mode in operation S460. For example, it is assumed that the skin care mode is an intensive trouble care mode and is a mode that the forehead portion of the user is intensively cared. In this example, The processor 130 may provide a light guide by emitting LEDs 510 corresponding to the user's forehead at less than a threshold intensity in a state in which the optical mask device 100 is not worn, as shown in FIG. 5, and when the mask device 100 is identified as being worn on the user's face, the processor 130 may control the optical mask device 100 to operate in an intensive trouble care mode. The processor 130 may control the LEDs 510 so that the LEDs 510 corresponding to the forehead portion of the user may emit light above or equal to the critical intensity for a time set in the intensive care mode.

If it is identified that the main body 110 is not worn on the user's face in operation S450, the light guide according to S430 may be kept maintained.

In this example, the processor 130 may emit the at least some LEDs by less than a threshold intensity by controlling a duty of a current provided to the at least some LEDs by less than a threshold value. The processor 130 may emit the at least some LEDs by greater than or equal to the threshold intensity by controlling the duty of the current provided to the at least some LEDs by greater than or equal to the threshold value.

The processor 130 may provide a light guide corresponding to the skin care mode by emission, by less than the threshold intensity, of at least some LEDs corresponding to the skin care mode, among the plurality of LEDs. The processor 130 may guide the skin care mode to the user by emitting the LED corresponding to the skin care mode to be applied to the user before the user wears the optical mask device 100. For example, if the skin care mode to be received by the user is a mode in which the red LED emits light, the red LED may emit light at a lower intensity than the light emitting intensity in the actual skin care mode and may provide information about the skin care mode to be used by the user.

The processor 130 may provide a light guide corresponding to the skin care mode by sequential emission, by less than the threshold intensity, of LEDs in different colors corresponding to the skin care mode, among the plurality of LEDs. For example, if a skin care mode to be received by a user emits a red LED for a first time and then sequentially emits a blue LED for a second time, the red LED may be emitted at an intensity lower than the intensity of the light emitting intensity in the actual skin care mode, and then the blue LED may be sequentially emitted to provide information on the skin care mode to be received by the user.

The processor 130 may sequentially emit different colors of LEDs during the time of providing the light guide based on a ratio of time when the LEDs of different colors are emitted in the actual skin care mode. For example, in the determined skin care mode, the LED in a first color may be emitted for a first time and the LED in a second color may be emitted for a second time that is shorter than the first time by greater than or equal to the threshold intensity. In this example, the processor 130 may provide the light guide so that an LED of the first color and an LED of the second color emit light by less than the threshold intensity, by a time corresponding to a ratio of the first time and the second time within a preset time. For example, if the skin care mode to be received by the user emits the red LED for 8 minutes, and then the blue LED is sequentially emitted for 4 minutes, the predetermined time, for example, 9 seconds may be divided into 2:1 and the red LED may be emitted for 6 seconds, and the blue LED may be sequentially emitted for 3 seconds to provide information about the skin care mode to be received by the user.

The time for providing the light guide may be pre-set at the time of manufacture, but may be set or changed by the user input. Alternatively, the time may be automatically set based on the user's usage history. For example, when, the user wears the optical mask device 100 within average 10 seconds after turning on the power of the optical mask device 100, the time for providing the light guide may be set to 10 seconds, and a light guide corresponding to the skin care mode may be provided within the corresponding time.

The processor 130 may provide the light guide described above only once, but may repeatedly provide the light guide while the user does not wear the optical mask device 100. For example, the processor 130 may repeatedly perform an operation of emitting a red LED for 6 seconds and sequentially emitting a blue LED for 3 seconds.

The processor 130 may determine a skin care mode to be received by the user as a mode corresponding to a user input, but may automatically determine a skin care mode regardless of a user input.

According to an example, the processor 130 may, based on the optical mask device being turned on, automatically determine the skin care mode which the user frequently uses. The processor 130 may automatically determine the skin care mode based on the usage history of the skin care mode if a separate user input is not received for selecting a skin care mode after the power of the optical mask device 100 is turned on.

According to another example, the processor 130 may automatically determine the skin care mode based on skin state information of the user received from an external device (e.g., the user terminal device 200 of FIG. 1). For example, the skin care mode may be automatically determined based on the corresponding information, by receiving information on an invisible region in which a trouble (e.g., acne) may be generated through skin measurement by UV light illumination or the like provided in the external device. Here, the external device may be implemented as a user terminal device 200 of FIG. 1, or an external measuring device (e.g., a smart mirror). For example, UV light may be installed in the smart mirror to enable skin condition analysis. In this example, one part of the optical mask device 100 may be included in a charging adapter, a remote controller, or the like.

According to another example, the processor 130 may determine a skin care mode to be used by the user based on information about the skin care mode received from an external device (e.g., the user terminal device 200 of FIG. 1).

According to another embodiment, the processor 130 may adjust a duty of a current provided to at least some LEDs based on a value sensed by a temperature sensor (not shown). For example, the processor 130 may, based on a value sensed by the temperature sensor during operation in the skin care mode being greater than or equal to a threshold temperature value, reduce the duty of the current provided to the at least some LEDs.

The processor 130 may control each LED by PWM control or continuously emit light depending on the characteristics of the LED color or type. For example, a blue LED may be PWM-controlled, and an NIR LED may be controlled by Fully ON (continuous wave). This is because the LED light sources of very short pulses may penetrate deeply within the cell tissue than in the case of continuous irradiation. In this example, a strong pulse of the first may make a cell tissue in an upper layer of the skin to be an excited state, and may serve to open a passage through which light may pass up to the cell tissue of the underlying layer.

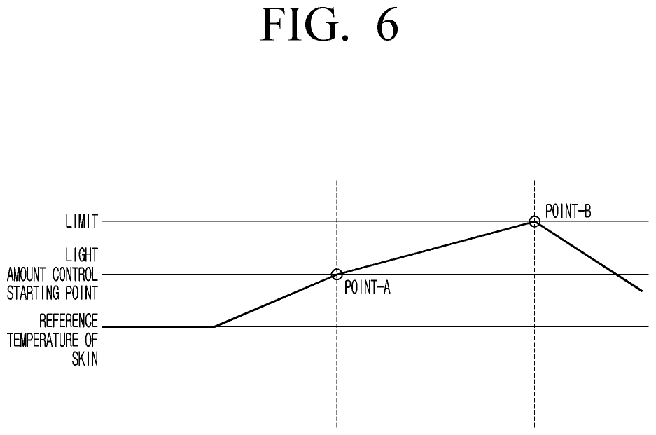

FIG. 6 is a diagram illustrating a method of controlling an LED according to skin temperature according to an embodiment of the disclosure.

According to an embodiment of the disclosure, the processor 130 may continuously measure the skin temperature based on the sensing value received from the temperature sensor during the skin care mode operation. In this example, the processor 130 may control the amount of light of the LED to be inversely proportional to the temperature change when the temperature change of the skin exceeds a (e.g., 1° C.) so that the optimal skin temperature for the skin care may be maintained.

For example, as shown in FIG. 6, when the skin temperature measured during the skin care mode operation on the basis of the skin temperature (reference skin temperature) measured at the start of the skin care mode reaches a threshold value (i.e., a preset light quantity control start point: point A), the duty of the LEDs emitted to reduce the current LED light quantity may be controlled. For example, the processor 130 may begin controlling the amount of light when the skin temperature is increased by 1° C. on the basis of the reference skin temperature. Specifically, as shown in FIG. 6, the PWM duty between point A to point B may be controlled to decrease to a predetermined value or gradually decrease. The threshold value for controlling the amount of light may be preset, or may be set or changed by the user. The threshold value may be automatically changed based on the usage history of the optical mask device 100, information received from the outside, and the like.

The skin care mode according to an embodiment may include at least one of a normal care mode or a speed care mode divided by a skin care time. The skin care mode may include at least one of an elasticity care mode, a trouble care mode, a whitening care mode, or a wrinkle care mode divided by a skin care type.

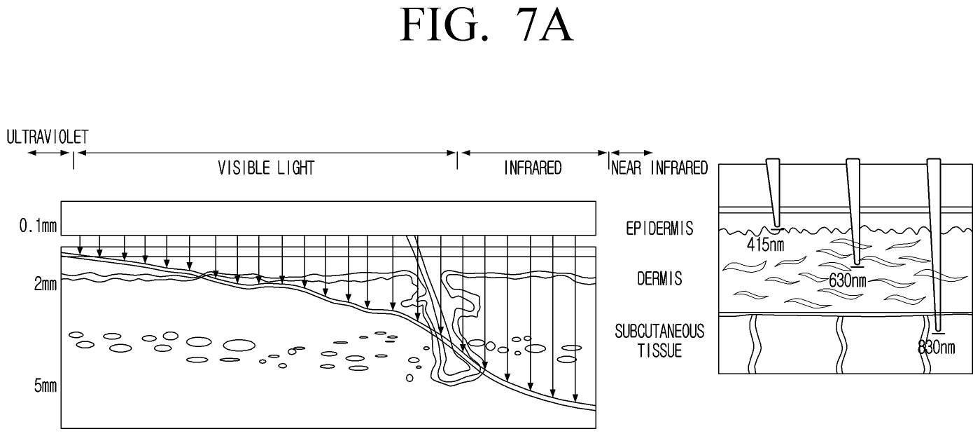

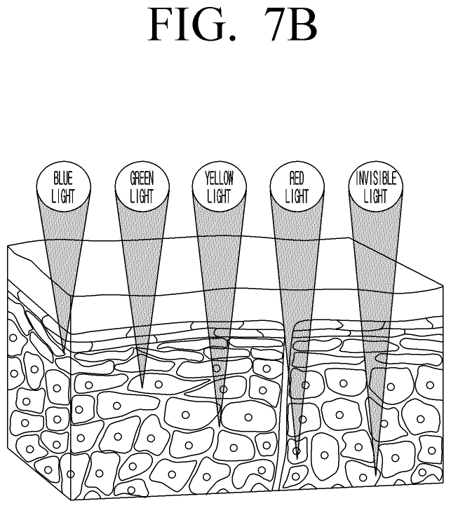

FIGS. 7A and 7B are diagrams to assist understanding of a skin care mode according to an embodiment of the disclosure.

FIG. 7A is a diagram illustrating a level of permeation into the skin by LED wavelengths; and FIG. 7B is a diagram illustrating a skin care effect by LED wavelengths.

For example, as shown in FIG. 7B, blue light (405 to 415 nm) may be helpful for sensitive skin and oily skin, and in particular, it may help to inhibit the proliferation of P.ACNE that causes acne. Green light (416 to 525 nm) may have the effect of improving skin tone and relieving wrinkles, especially by removing toxins from the skin to revitalize the skin and help to soothe the skin. Yellow light (526 to 590 nm) may be effective in suppressing melanin production and improving liver spots/freckles, and particularly may help treat face redness by improving lymph and blood circulation. In addition, red light (630 to 660 nm) is a light that penetrates to the dermis of the skin and promotes the production of elastin and collagen, which may help skin elasticity and regeneration. Near-infrared (IR) rays (800 to 900 nm) may be irradiated deeply up to 4 nm of the skin, and may prevent skin pigmentation and promote skin regeneration. However, IR rays are generating heat so irradiating IR rays for a long time may be avoided.

Based on the effect of FIG. 7A and FIG. 7B, the LED color applied to the various skin care modes according to an embodiment may be determined.

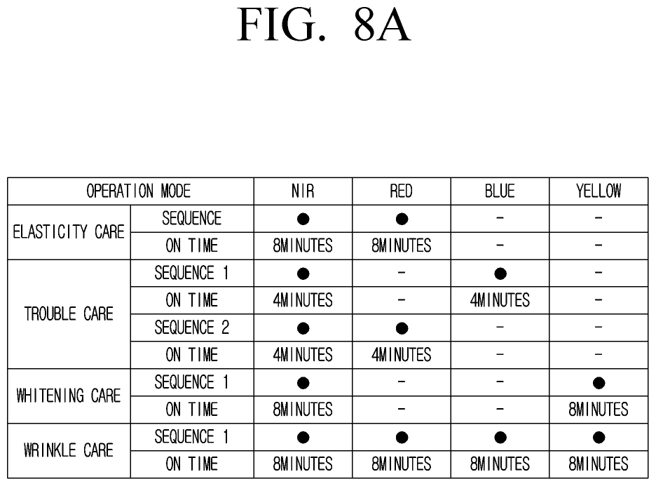

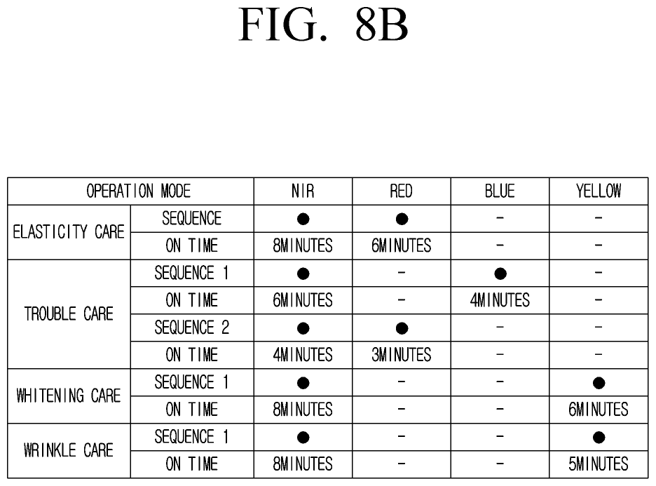

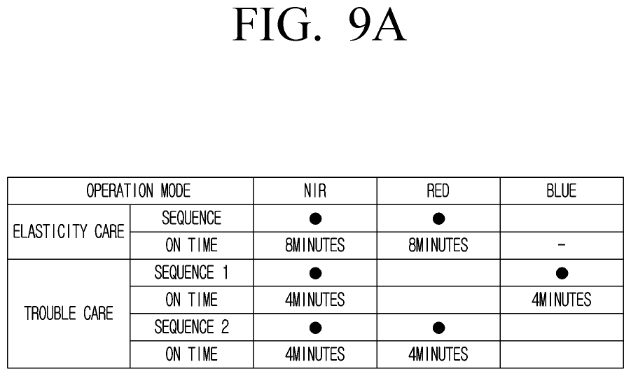

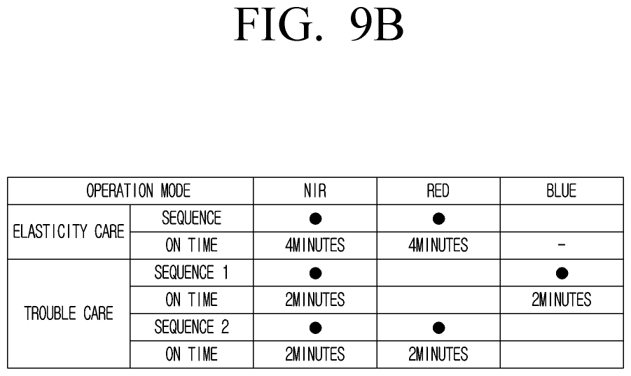

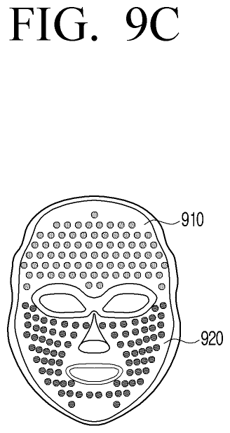

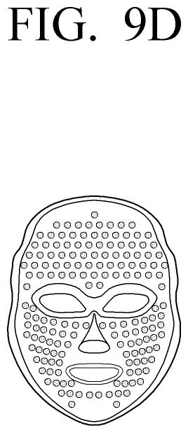

FIGS. 8A, 8B, and 9A, 9B, 9C and 9D are diagrams illustrating various skin care modes according to an embodiment of the disclosure.

FIG. 8A illustrates an example of a normal care mode according to an embodiment and FIG. 8B illustrates an another embodiment of a normal care mode.

According to an embodiment of the disclosure, as shown in FIG. 8A, the processor 130 may sequentially irradiate different types of LEDs for each skin care mode based on the skin care effect of each LED, but each LED operation time may be controlled equally. For example, the processor 130 may differently control the driving time for each red/blue/yellow/color combination (violet, pink, etc.) in each skin care mode as shown in FIG. 8A and may provide an LED light suitable for each skin care mode.

According to another embodiment of the disclosure, as shown in FIG. 8A, the processor 130 may sequentially irradiate different types of LEDs according to each skin care mode based on the skin care effect of each LED, and may control the operation time differently. For example, as shown in FIG. 8A, the processor 130 may provide LED light suitable for each skin care mode by differently controlling the operation time for each of red/blue/yellow/color combinations (violet, pink, etc.) in each skin care mode.

FIG. 9A illustrates an LED light-emitting time in an elasticity care mode and a trouble care mode in a general skin care mode according to an embodiment of the disclosure, and FIG. 9B illustrates an LED light-emitting time in an elasticity care mode and a trouble care mode in a speedy mode.

According to an embodiment of the disclosure, in the normal care mode, the processor 130 may emit LEDs by a predetermined operating time based on a specific intensity (or light intensity) for each skin care mode, as shown in FIG. 9A. For example, the processor 130 may emit the NIR LED for 8 minutes based on 100% of the amount of light required in the elasticity care mode, and then emit Red LED for 8 minutes to perform a care for a total of 16 minutes.

Meanwhile, in the speedy mode, the processor 130 may emit LEDs by shortening the operation time, though the accumulated light amount as in the normal care mode is the same as the normal care mode as shown in FIG. 9B. For example, the processor 130 may emit NIR LEDs for 4 minutes based on twice the amount of light required in the elasticity care mode, that is, 200% light amount, and then emits RED LEDs for 4 minutes to provide care for a total of 8 minutes. However, in this example, although the operating time is shortened than the time shown in FIG. 9A, the PWM duty may be doubled so that the accumulated light quantity becomes the same as the normal care mode shown in FIG. 9A.

FIG. 9C illustrates an LED emission type in an elasticity care mode and FIG. 9D illustrates an LED emission type in a trouble care mode.

According to an embodiment of the disclosure, as shown in FIG. 9C, in the elasticity care mode, all LEDs provided in the optical mask device 100 may emit light with uniform intensity, but as shown in FIG. 9D, in the trouble care mode, only an LED 910 corresponding to the trouble location may be emitted, and the remaining LEDs 920 may not be emitted. Alternatively, the LED 910 corresponding to the trouble location may emit light with a first intensity, and the remaining LEDs 920 may emit light with a second intensity. Alternatively, the first type LED 910 may be emitted in the trouble area, and the second type of LED 920 may be emitted in the remaining area.

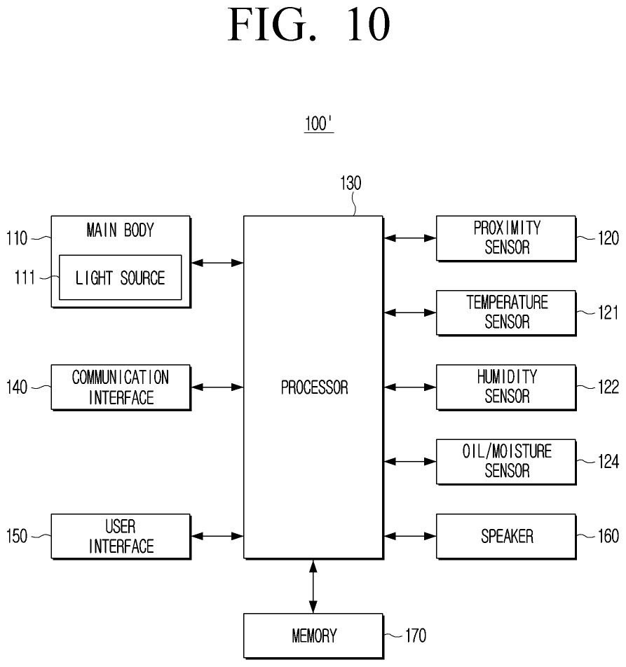

FIG. 10 is a block diagram illustrating a specific configuration of an optical mask device according to an embodiment of the disclosure.

Referring to FIG. 10, an optical mask device 100′ may include the main body 100, a proximity sensor 120, a temperature sensor 121, a humidity sensor 122, an oil/moisture sensor 124, a processor 130, a communication interface 140, a user interface 150, a speaker 160, and a memory 170. Among the configurations shown in FIG. 10, detailed descriptions of configurations overlapping those shown in FIG. 2 will be omitted. In addition, some of the components shown in FIG. 10 in the optical mask device 100′ may be omitted.

The temperature sensor 121 is provided in one area of the optical mask device 100, and may measure the temperature of the skin. For example, the temperature sensor 121 may be implemented as at least one of a contact type temperature sensor and a non-contact type temperature sensor.

The humidity sensor 122 is provided in one area of the optical mask device 100, and may measure the moisture level of the skin. The humidity sensor 122 may be implemented as a wet/dry humidity sensor, an electrolytic humidity sensor, a polymer film humidity sensor, a ceramic humidity sensor, a thermistor humidity sensor, a microwave humidity sensor, a condensation sensor, or the like.

The oil/moisture sensor 124 may be provided in one area of the optical mask device 100 and may measure the oil/moisture level of the skin.

The communication interface 140 may include a communication circuitry and may communicate with various external devices (e.g., a user terminal device 200, a server, etc.). For example, the communication interface 140 may receive diverse data from an external device (e.g., a source device), an external storage medium (e.g., a universal serial bus (USB) memory), an external server (e.g., a webhard) through communication methods such as, for example, and without limitation, an access point (AP)-based wireless fidelity (Wi-Fi) (wireless local area network (WLAN)), Bluetooth, Zigbee, wired/wireless local area network (LAN), wide area network (WAN), Ethernet, 5th generation (5G), IEEE 1394, high definition multimedia interface (HDMI), universal serial bus (USB), mobile high-definition link (MHL), advanced encryption standard (AES)/European broadcasting union (EBU), optical, coaxial, or the like.

The user interface 150 may be implemented as a button, a touch pad, or a device such as a mouse, or may be implemented as a touch screen, a remote controller transceiver, etc., which may perform a display function and an operation input function as well. For example, the button may be various types of buttons such as at least one of a mechanical button, a touch pad, a wheel, or the like, formed in an arbitrary area such as at least one of a front portion, a side portion, a back portion, or the like, of the outer surface of the main body of the optical mask device 100′. For example, a button for turning on/off the power of the optical mask device 100′ may be provided. The remote control transceiver may receive a remote control signal from an external remote control device or transmit a remote control signal through at least one of infrared communication, Bluetooth communication, and Wi-Fi communication.

The speaker 160 may output various audio data processed by the processor 130, as well as various notification sound, voice messages, or the like.

The memory 170 may store data necessary for various embodiments. The memory 170 may be implemented as at least one of a memory embedded within the optical mask device 100 or a memory detachable from the optical mask device 100 according to the usage of data storage. For example, the data for driving the optical mask device 100 may be stored in the memory embedded within the optical mask device 100, and the data for upscaling of the optical mask device 100 may be stored in the memory detachable from the optical mask device 100. A memory embedded in the optical mask device 100 may be implemented as at least one of a volatile memory such as a dynamic random access memory (DRAM), a static random access memory (SRAM), a synchronous dynamic random access memory (SDRAM), or anon-volatile memory (e.g., one time programmable ROM (OTPROM), programmable ROM (PROM), erasable and programmable ROM (EPROM), EEPROM, mask ROM, flash ROM, a flash memory (e.g., NAND flash or NOR flash), a hard disk drive (HDD), a solid state drive (SSD), or the like. A memory detachably mounted to the optical mask device 100 may be implemented as a memory card (e.g., a compact flash (CF), a secure digital (SD), micro secure digital (micro-SD), a mini secure digital (mini-SD), an extreme digital (xD), a multi-media card (MMC), etc.), an external memory (e.g., a universal serial bus (USB) memory, or the like) connectable to the USB port, or the like.

According to an example, the memory 170 may store various information related to the operation of the optical mask device 100 such as information about the history of use of the optical mask device 100, for example, information related to the history of use of the user's skin care mode, information related to the user's skin condition, information related to LED driving in the skin care mode, or the like.

The power supplier 180 serves to supply power to the optical mask device (100). For example, the power supply unit 180 may supply power by charging a rechargeable battery. In this example, the optical mask device 100 may guide the charging situation through the light of the LED lamp provided in the charger.

The optical mask device 100′ may further include a camera, a microphone, a global positioning system (GPS) receiver, or the like.

According to an embodiment of the disclosure, the optical mask device 100′ may transmit information on the skin care mode selected by the user to an external device, determine a care mode preferred by a user by learning by an external device the received information using the AI model, and may transmit the information to the optical mask device 100′.

Alternatively, the external device may continuously learn information about the user's skin condition obtained through a camera, etc. using an AI model to determine information on a skin care mode suitable for the user, and may transmit the information to the optical mask device 100′.

The external device may be implemented with an external server, the user terminal device 200 of FIG. 1 or the like.

According to the above-described various embodiments, it is possible to provide an optimized care mode to the user by differently controlling the type of LED, the amount of LED light, the LED irradiation time, and the driving method according to the skin diagnosis result of each user.

In addition, while providing information about the skin care mode to the user by using the LED, it is possible to minimize the power consumption of the LED before wearing the mask.

Various embodiments of the disclosure may be applied not only to an optical mask device but also to any electronic device capable of performing skin care using light.

The above-described methods according to various embodiments of the disclosure may be implemented in the form of an application or software that may be installed in an existing optical mask device or a user terminal device. Alternatively, the methods according to various embodiments of the disclosure described above may be performed using a deep learning-based artificial neural network (or deep artificial neural network), that is, a learning network model.

In addition, the methods according to various embodiments may be implemented only with software upgrade or hardware upgrade for the conventional optical mask device or user terminal device.

Various embodiments of the disclosure described above may be performed through an embedded server or an external server provided in an existing optical mask device or a user terminal device.

The various embodiments described above may be implemented as software including instructions stored in a machine-readable storage media which is readable by a machine (e.g., a computer). The device may include an image processing device (e.g., image processing device A) according to the disclosed embodiments, as a device which calls the stored instructions from the storage media and which is operable according to the called instructions. When the instructions are executed by a processor, the processor may directory perform functions corresponding to the instructions using other components or the functions may be performed under a control of the processor. The instructions may include code generated or executed by a compiler or an interpreter. The machine-readable storage media may be provided in a form of a non-transitory storage media. The ‘non-transitory’ means that the storage media does not include a signal and is tangible, but does not distinguish whether data is stored semi-permanently or temporarily in the storage media.

According to an embodiment of the disclosure, the method according to the various embodiments described herein may be provided while being included in a computer program product. The computer program product may be traded between a seller and a purchaser as a commodity. The computer program product may be distributed in the form of a machine-readable storage medium (e.g.: a compact disc read only memory (CD-ROM)), or distributed online through an application store (e.g.: PLAYSTORE™). In the case of online distribution, at least a portion of the computer program product may be at least temporarily stored in a storage medium such as a server of a manufacturer, a server of an application store, or a memory of a relay server, or temporarily generated.

Each of the elements (e.g., a module or a program) according to various embodiments may be comprised of a single entity or a plurality of entities, and some sub-elements of the abovementioned sub-elements may be omitted, or different sub-elements may be further included in the various embodiments. Alternatively or additionally, some elements (e.g., modules or programs) may be integrated into one entity to perform the same or similar functions performed by each respective element prior to integration. Operations performed by a module, a program, or another element, in accordance with various embodiments, may be performed sequentially, in a parallel, repetitively, or in a heuristically manner, or at least some operations may be performed in a different order, omitted or a different operation may be added.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An optical mask device comprising:
a main body having a face shape and wearable on a face of a user;
a plurality of light-emitting diode (LED) light sources provided in the main body;
a proximity sensor to send a signal in response to sensing a presence of the face of the user; and
a processor configured to:
receive a skin care mode selected by the user;
identify whether the main body is worn on the face of the user based on the signal received from the proximity sensor; and
in response to the identifying that the main body is not worn on the face of the user, provide a light guide corresponding to the skin care mode by controlling at least some LEDs corresponding to the skin care mode among a plurality of LEDs to emit an intensity less than a threshold intensity.

2. The optical mask device of claim 1, wherein the processor is further configured to, in response to the identifying that the main body is worn on the face of the user while providing the light guide, control the optical mask device to operate in the skin care mode by emitting the at least some L with an intensity greater than or equal to the threshold intensity.

3. The optical mask device of claim 2, wherein the processor is further configured to:
emit the at least some LEDs with the intensity less than the threshold intensity by controlling a duty of a current provided to the at least some LEDs with a value less than a threshold value; and
emit the at least some LEDs with the intensity greater than or equal to the threshold intensity by controlling the duty of the current provided to the at least some LEDs with a value greater than or equal to the threshold value.

4. The optical mask device of claim 3, further comprising:
a temperature sensor,
wherein the processor is further configured to, based on a value, sensed by the temperature sensor while operating in the skin care mode, being greater than or equal to a threshold temperature value, reduce the duty of the current provided to the at least some LEDs.

5. The optical mask device of claim 1, wherein the processor is further configured to provide a light guide corresponding to the skin care mode by emitting the at least some LEDs corresponding to the skin care mode among the plurality of LEDs with the intensity less than the threshold intensity.

6. The optical mask device of claim 5, wherein the processor is further configured to provide a light guide corresponding to the skin care mode by sequentially emitting LEDs in different colors corresponding to the skin care mode among the plurality of LEDs with the intensity less than the threshold intensity.

7. The optical mask device of claim 6, wherein the processor is further configured to, based on emission, by greater than or equal to the threshold intensity, of the LED in a first color for a first time in the skin care mode and emission, by greater than or equal to the threshold intensity, of the LED in a second color for a second time that is shorter than the first time, provide the light guide so that an LED of the first color and an LED of the second color emit light by less than the threshold intensity, by a time corresponding to a ratio of the first time and the second time respectively within a preset time.

8. The optical mask device of claim 1, further comprising:
a memory storing usage history of a skin care mode by the user,
wherein the processor is further configured to, based on the optical mask device being turned on, automatically determine the skin care mode based on the stored usage history of a skin care mode of the user.

9. The optical mask device of claim 1, wherein the processor is further configured to automatically determine the skin care mode based on skin state information of the user received from an external device.

10. The optical mask device of claim 1, wherein the skin care mode comprises a normal care mode or a speedy care mode divided by a skin care time.

11. The optical mask device of claim 1, wherein the skin care mode comprises at least one of an elasticity care mode, a trouble care mode, a whitening care mode, or a wrinkle care mode divided by a skin care type.

12. A control method of an optical mask device comprising a main body having a face shape and wearable on a face of a user, a plurality of light-emitting diode light sources provided in the main body, and a proximity sensor to send a signal in response to sensing a presence of the face of the user, the method comprising:
determining a skin care mode for the face of the user;
identifying whether the main body is worn on the face of the user based on the signal received from the proximity sensor; and
in response to the identifying that the main body is not worn on the face of the user, providing a light guide corresponding to the skin care mode by controlling at least some LEDs corresponding to the skin care mode among a plurality of LEDs to emit an intensity less than a threshold intensity.

13. The method of claim 12, further comprising:
in response to the identifying that the main body is worn on the face of the user, operating in the skin care mode by emitting the at least some LEDS with an intensity greater than or equal to the threshold intensity.

14. The method of claim 13, wherein the providing of the light guide further comprises emitting the at least some LEDs with the intensity less than the threshold intensity by controlling a duty of a current provided to the at least some LEDs with a value less than a threshold value, and
wherein the operating in the skin care mode further comprises emitting the at least some LEDs with the intensity greater than or equal to the threshold intensity by controlling the duty of the current provided to the at least some LEDs with a value greater than or equal to the threshold value.

15. The method of claim 14, wherein the optical mask device further comprises a temperature sensor, and the method further comprises, based on a value, sensed by the temperature sensor while operating in the skin care mode, being greater than or equal to a threshold temperature value, reducing the duty of the current provided to the at least some LEDs.

16. The method of claim 12, the providing of the light guide comprises providing a light guide corresponding to the skin care mode by emitting the at least some LEDs corresponding to the skin care mode among the plurality of LEDs with the intensity less than the threshold intensity.

17. The method of claim 16, the providing of the light guide comprises providing a light guide corresponding to the skin care mode by sequentially emitting LEDs in different colors corresponding to the skin care mode among the plurality of LEDs with the intensity less than the threshold intensity

18. The method of claim 17, the providing of the light guide comprises, based on emission, by greater than or equal to the threshold intensity, of the LED in a first color for a first time in the skin care mode and emission, by greater than or equal to the threshold intensity, of the LED in a second color for a second time that is shorter than the first time, providing the light guide so that an LED of the first color and an LED of the second color emit light by less than the threshold intensity, by a time corresponding to a ratio of the first time and the second time respectively within a preset time.

19. The method of claim 12, further comprising:
based on the optical mask device being turned on, automatically determining the skin care mode based on the stored usage history of a skin care mode of the user.

20. The method of claim 12, further comprising:
automatically determining the skin care mode based on skin state information of the user received from an external device.
